(19) United States
(12) Patent Application Publication
(10) Pub. No.: US 2015/0307952 A1
(43) Pub. Date: Oct. 29, 2015

(54) LIQUID CO-EXTRACTION PROCESS FOR PRODUCTION OF SUCROSE, XYLO-OLIGOSACCHARIDES AND XYLOSE FROM FEEDSTOCK

(71) Applicant: BRADLEY A. SAVILLE, Oakville (CA)

(72) Inventor: BRADLEY A. SAVILLE, Oakville (CA)

(21) Appl. No.: 14/699,547

(22) Filed: Apr. 29, 2015

Related U.S. Application Data
(60) Provisional application No. 61/985,498, filed on Apr. 29, 2014.

(51) Int. Cl. C13B 10/00; C13K 11/00; C13K 1/02; C12P 7/02

(52) U.S. Cl. CPC C13B 10/00; C12P 7/02; C13K 11/00; C13K 1/02

(57) ABSTRACT

The present invention provides methods for extraction of sucrose, xylo-oligosaccharides, xylose and bioactive compounds from feedstock, and in particular to the hot-water co-extraction of sucrose, fructose, glucose, xylo-oligosaccharides, or xylose and bioactive compounds from feedstock.

### CROSS-REFERENCE TO RELATED APPLICATIONS

This application claims priority under 35 USC 119(e) from U.S. Provisional Patent Application No. 61/985,498 filed Apr. 29, 2014, which is hereby incorporated by reference in its entirety.

### STATEMENT REGARDING FEDERALLY SPONSORED RESEARCH OR DEVELOPMENT

Not applicable.

### FIELD OF THE INVENTION

The present invention generally relates to the extraction of sucrose, glucose, fructose, xylo-oligosaccharides, xylose and bioactive compounds from feedstock and, in particular, to the liquid co-extraction of sucrose (fructose, glucose), xylo-oligosaccharides, xylose and bioactive compounds from feedstock.

### BACKGROUND OF THE INVENTION

Lignocellulosic biomass has been developed as a non-food source of feedstock for biofuel ethanol production. Lignocellulosic biomass may be classified into four main categories: (1) wood residues (sawdust, bark or other), (2) municipal solid waste, (3) agricultural residues (including corn stover, corncobs and sugarcane bagasse), and (4) dedicated energy crops (which are mostly composed of fast growing, tall, woody grasses such as sugarcane, energycane, switchgrass and Miscanthus).

Lignocellulosic biomass comprises three primary polymers that make up plant cell walls: Cellulose, hemicellulose, and lignin. Cellulose fibres are locked into a rigid structure of hemicellulose and lignin. Lignin and hemicelluloses form chemically linked complexes that bind water soluble hemicelluloses into a three dimensional array, cemented together by lignin. Lignin covers the cellulose microfibrils and protects them from enzymatic and chemical degradation. These polymers provide plant cell walls with strength and resistance to degradation, which makes lignocellulosic biomass a challenge to use as substrate for biofuel production. Lignocellulosic biomass may also contain other components such as extractives, organic acids, and ash.

Currently, much of the ethanol in the world is produced via so-called first generation (1G) processes. One common method is to press, diffuse or mill a sugar-rich feedstock such as sugarcane, and thereby extract the water and sucrose from the feedstock, and then ferment the sucrose to ethanol and distill. This fibrous residue remaining after removing the sucrose by crushing, pressing and/or milling the feedstock is called bagasse. A different 1G method, primarily employed in North America, involves enzymatic conversion of starch into sugars, which are then fermented into ethanol and distilled. The sugars obtained from these processes may also be used as feedstocks to make other biofuels, such as butanol or hydrocarbons. These sugars may also be fed to systems that produce algae from heterotrophic organisms, or may be used as the carbon source for production of other biochemicals, bioproducts or pharmaceuticals using fermentation or catalytic technologies. A typical 1G process for production of ethanol from sugarcane is illustrated in FIG. 1.

As illustrated in FIG. 1, these 1G technologies generally considered the bagasse to be a waste and disregarded the bagasse component as a source of fermentable sugars. More recently, bagasse has become regarded as a valuable source of feedstock for cellulosic ethanol and cellulose-derived biochemical production. As such, second-generation (2G) processes have been developed to produce sugars through the use of lignocellulosic feedstocks that contain cellulose, hemicellulose, and lignin. Typically, in these processes, the complex carbohydrates in cellulose and hemicellulose are broken down into glucose and xylose, respectively, via a complex process involving transfer of material during this several step process of biomass pretreatment and enzymatic hydrolysis. The more rigid β-1,4 linkage between glucose units in cellulose means that aggressive pre-processing/pre-treatment is required before these feedstocks can be hydrolyzed into simple sugars. Pre-processing is achieved via pretreatment, which typically involves some combination of chemical, biological, mechanical, and thermal treatment steps to disrupt the bonds between cellulose, and lignin, while activating the cellulose and making it more susceptible for enzymatic conversion. In contrast, sugars from xylan and other hemicellulose components are known to be easier to release and solubilize from the biomass structure.

Prior art biomass pretreatment processes involve treatment steps that degrade soluble sugars. For example, steam explosion pretreatment, or “autohydrolysis”, is typically conducted at temperatures between 190° and 230° C., either with our without chemical catalysts. If an acid catalyst is used in conjunction with the steam, the temperature may be reduced to 170° or 175° C. These high temperatures (170°-230° C.) are necessary to fully disrupt the bond structure within and between cellulose fibrils, but unfortunately, can also lead to destruction of soluble sugars produced from xylan and other hemicellulose components. The presence of an acid catalyst increases the degree and rate of degradation of these soluble sugars. Degradation products of xylose and fructose, typically furfural and hydroxyl-methyl furfural (5-HMF), are known to be potent inhibitors of downstream fermentation processes. Thus, the choice of process conditions has previously involved a trade-off between cellulose activation, which requires high temperatures, and xylan/xylose preservation, which requires lower temperatures.

The issue with soluble sugar degradation and inhibitor production is of particular concern with 2G bagasse feedstocks that may contain residual sucrose, such as sugar cane, energy cane, Miscanthus, sweet sorghum and sugar beets. The bagasse fraction may comprise the stalks, or the stalks, tops and leaves. Sugarcane, energy cane, and sweet sorghum, for example, have high concentrations of stem sucrose. However, the majority of carbohydrate in cane is lignocellulosic.

Prior art 2G technologies developed to process sugarcane bagasse and energy cane bagasse are based on the premise that a very high percentage of the soluble sucrose has been extracted as “juice” in the milling or cutting of the stalks, prior to processing the residual bagasse with a second generation technology that targets the cellulose and hemicellulose in these residues. However, without juice extraction, bagasse feedstock may contain appreciable levels of soluble sugars that hydrolyze under heat and/or acid to form fermentation inhibitors. For example, sucrose yields glucose and fructose, and fructose/glucose form 5-HMF. 5-HMF is a potent fermentation inhibitor, consequently allowing free sugar such as the sucrose present in the juice to enter the high temperature pretreatment system is considered a liability. This transformation to 5-HMF also directly impacts the production of ethanol or other biochemicals because the ethanol (or other product) equivalents of the fructose are lost to degradation in the high temperature pretreatment process. Clearly, allowing soluble sugars to continue into a conventional pretreatment process is considered to be a serious disadvantage, and it is desired to put process steps in place to limit this possibility.

Conventional prior art approaches to address this challenge have been to develop separate processes for juice extraction and for processing of bagasse to use as feedstock. For example, feedstock is harvested, milled and subject to countercurrent washing with water to remove as much of the water (“juice”) content as possible. The extracted juice is clarified and fermented to alcohol in a clear liquid fermentation, and the bagasse (essentially free of soluble sucrose) is pretreated in a 2G process. Known 2G processes employ a variety of different pretreatment technologies, but all currently employ the high temperatures that are known to be destructive to xylan/xylose and other hemicellulose components as well as residual sucrose-derived carbohydrates.

In order to address these limitations, separate pretreatment prior art processes have been derived, to perform hemicellulose solubilization in a first, lower temperature process, followed by cellulose activation in a second, higher temperature process. For example, a liquid extraction step can generate a pentose (C5)-rich stream from hemicellulose that ultimately is converted into a molasses stream for animal feed or other applications. Other liquid extraction methods have been proposed, for example a system for extraction of hemicellulosic sugars under a range of temperatures, between 180° to 185° C. (U.S. Pat. Pub. 20100116267). In other processes, the first stage of pretreatment is followed by a second stage that focuses on cellulose activation; for example, Yu et al. recommended conditions for this stage as 200° C. for 20 min. Yu et al. (Bioresource Technology, 101, 4895-4899, 2010)

In summary, to date, various 2G studies and technologies have aimed to process lignocellulosic biomass by including extraction of hemicellulose at temperatures between 170° C. (with catalyst) and 185° C., with retention times between 15 and 60 minutes. These processes have been designed in an attempt to limit degradation of xylose to furfural while obtaining a high yield of xylose and xylo-oligosaccharides from hemicellulose. This range of temperatures and retention times, however, are clearly destructive to soluble sugars in feedstocks, especially those containing significant amounts of sucrose or fructose. As a result, when lignocellulosic biomass, is processed/pretreated by conventional methods described in the art, a portion of the soluble sugars do not contribute to the yield of biofuel, and furthermore they generate inhibitors of downstream fermentation.

Therefore, in order to obtain an improved technically and economically viable conversion process for soluble sugar production for use in industry such as biofuels, the maximum utilization of all sugar fractions of feedstocks is desired. The present invention meets this need through a co-extraction process that removes sucrose along with the xylo-oligosaccharides and xylose in the sucrose-laden feedstock through a primarily liquid water extraction process. The process enhances extraction efficiency of soluble sugars from feedstocks, minimizes degradation of sugars and minimizes the formation of inhibitory compounds, while reducing capital costs and improving process efficiency. The inventive process also co-extracts soluble bioactive compounds and low-molecular weight extractives and polyphenols derived from the lignin and lignocellulosic biomass.

### BRIEF DESCRIPTION OF THE DRAWINGS

FIG. 1 is a prior art conventional process for production of sugars and bio-products from sucrose-rich feedstocks.

FIG. 2 is the inventive process for production of sugars and bio-products from sucrose-rich feedstocks.

FIG. 3 shows a crushing process coupled with inventive process of FIG. 2.

FIG. 4 illustrates the percent xylo-oligosaccharides, relative to dry weight of biomass, extracted in the inventive processes by temperature and time.

FIG. 5 illustrates the percent acetic acid produced, relative to dry weight of biomass, in the inventive processes by temperature and time.

FIG. 6 illustrates the percent furfural produced, relative to dry weight of biomass, in the inventive processes by temperature and time.

FIG. 7 illustrates the percent xylo-oligosaccharides, relative to total xlyan content of biomass, extracted in the inventive processes by temperature and time.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice of the present invention, the preferred materials and methods are described herein. Accordingly, the terminology used herein is for the purpose of describing particular embodiments only, and is not intended to be limiting.

As used herein, the term “bagasse” means the fibrous residue remaining after any of the various feedstocks are crushed and their juice extracted.

As used herein, the term “bioactive compound” means compounds in crops and foods that may confer health benefits beyond their nutrient content, for example, by interacting with enzymes, proteins or cells or affecting cell metabolism, or affecting the presence or activity of oxidative compounds or free radicals. Examples include polyphenolic compounds, tannins, flavonoids, extractives, anthocyanins, isoflavones, catechins, and soluble oiligosaccarides.

As used herein, “bio-fuel” means a liquid or solid fuel produced from renewable feedstocks.

As used herein, “bio-product” means materials, chemicals and energy derived from renewable biological sources.

As used herein, “C5 sugar” means sugars that have a five carbon backbone. For example, C5 sugars are sugars such as xylose, and its corresponding oligomeric and polymeric forms that contain multiple C5 units.

As used herein, “C6 sugar” means sugars that have a six carbon backbone. For example, glucose is a common six carbon sugar. This also includes oligosaccharides that contain multiple C6 units.

As used herein, the term “juice” means the water-based liquid that is extracted from sugar-rich feedstock upon physical treatment, for example, without limitation, milling with a roller mill.

As used herein the term “physical processing” and “physically processing” of feedstock mean a physical treatment that breaks, shreds and/or crushes the structure of lignocellulosic materials thereby reducing the particle size and increasing surface area; for example milling, cutting and/or pressing feedstock.

As used herein, the term “pressure vessel” is defined as a vessel in which the pressure is obtained from an indirect source or by the application of heat from an indirect source or a direct source.

As used herein, “refining” means a physical process employing shearing devices or plates to reduce fiber/particle size and remove fibrils.

As used herein, “saturation pressure conditions” means the pressure of a vapor which is in equilibrium with its liquid (as steam with water), specifically, the maximum pressure possible by water vapor at a given temperature.

As used herein, the term “soluble hemicellulosic sugars” means low molecular weight carbohydrates, including monomers, oligomers and polymers derived from 5-carbon monomer units, that are able to dissolve in a liquid medium under extraction conditions.

As used herein, “soluble sugars” means low molecular weight carbohydrates, including monomers, dimers, trimers, and other oligosaccharides that will freely dissolve in the medium/solvent under extraction conditions.

As used herein, the term “steam explosion” means a treatment to solubilize hemicellulose to oligosaccharides and/or activate cellulose, which takes place at temperatures between 160° and 240° C. and pressure between 0.7 and 4.8 MPa, and includes a rapid decompression step that reduces the original size of the feedstock material.

As used herein, the term “intact feedstock” refers to a feedstock containing soluble sugars, polymeric carbohydrates such as cellulose and hemicellulose, lignin and other extractives, that has not been processed to remove any of these compounds.

As used herein, the term “sucrose-derived extracts” refers to soluble sugar mixtures that may contain glucose and/or fructose derived from sucrose hydrolysis, and may optionally also include intact sucrose.

### BRIEF SUMMARY OF THE INVENTION

The present invention is directed to methods which maximize the extraction yield of soluble sugars from lignocellulosic feedstocks, particularly from feedstocks that include sucrose, hemicellulosic and cellulosic sugars. More specifically, the invention concerns an extraction process during which sucrose (fructose, glucose) is co-extracted along with soluble hemicellulosic sugars, such as xylo-oligosaccharides and xylose. Extractives and organic acids in the biomass and bioactive compounds derived from lignin may also be solubilized and co-extracted through use of the inventive method. A significant advantage of the method of the present invention over the prior art is that extraction of sucrose and xylose occurs with less production of furfural and 5-HMF. A further advantage is efficiency and ease of processing in a single cook vessel. An exemplary feedstock, without intent to be limiting, is raw energy cane which typically contains about 5-8 wt % soluble sucrose (wet basis) and about 50-70 wt % carbohydrate (dry basis), including hemicellulosic and cellulosic sugars. The invention is also directed to the products of such methods.

Thus, in one aspect, the present invention provides methods in which lignocellulosic feedstocks are processed in a hot water co-extraction step to extract sucrose (which may be present in intact form and/or as sucrose hydrolyzed to fructose and glucose) and soluble hemicellulosic sugars. The hot water co-extraction is performed without addition of strong acids or alkalis for solvent extraction, ionic liquids, glycerol, organic solvents, or other exogenous chemicals. The methods can be applied to various lignocellulosic feedstocks containing sugars, including, but not limited to, energy cane, sugar cane, sugar beets, sweet sorghum, fiber sorghum and miscane. The feedstocks may be provided in any form including, but not limited to, raw, green cut, dry or partially dry, straw, billet material and/or bagasse. Preferably, the feedstock includes at least approximately 1 wt. % or more sucrose, more preferably the feedstock includes approximately 3 wt. % or more sucrose, even more preferably the feedstock includes approximately 5.5 wt. % or more sucrose, and most preferably the feedstock includes approximately 8 wt. % or more sucrose. The feedstock may also contain in the range of up to 30 wt. %.

Water is added to the feedstock in a volume sufficient to keep the water primarily in the liquid state during the extraction process. The inventive co-extraction method is performed on feedstocks preferably at a range of extraction temperatures of between 120° C. and 180° C., more preferably at temperatures between 150° C. and 170° C. The feedstock is held at the extraction temperatures for a predetermined time (“retention time”), preferably a retention time of from approximately 30 minutes to 8 hours, more preferably from approximately 30 minutes to 3.5 hours, most preferably from approximately 30 minutes to 2 hours. The extraction process ultimately produces a liquids stream and a solids stream which are eventually separated, both of which can be further processed according to conventional methods known in the art.

In one embodiment of this aspect, extraction may be performed under increased pressure, and the temperature and/or pressure may be varied during the extraction process.

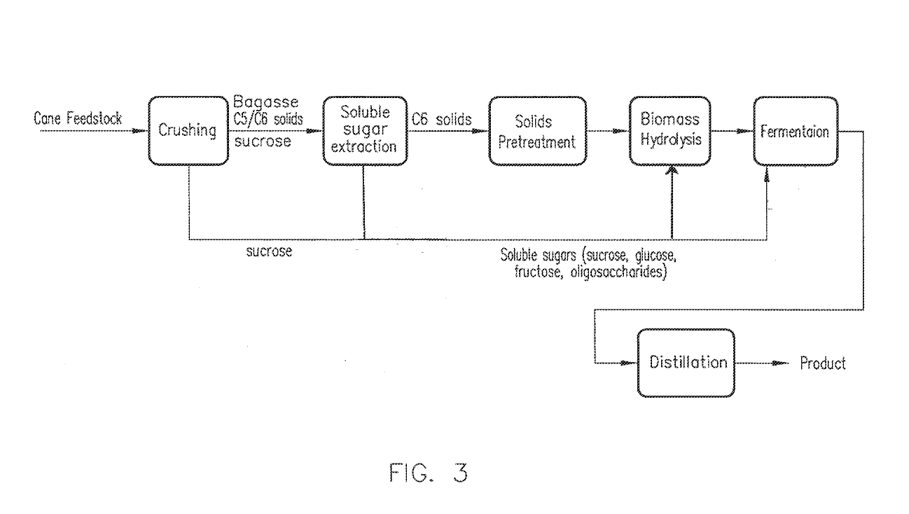

In another embodiment of this aspect, the method optionally includes physical processing, of feedstock for example by conventional milling, cutting, pressing or other methods to physically reduce the particle size and increase the surface area of the feedstock. Physical processing can take place with or without water being added to induce imbibition of the feedstock. Such methods may be performed before (FIG. 3) and/or after the hot water co-extraction process of the present invention. The liquid generated during such physical processes contains varying amounts of sucrose or sucrose-derived carbohydrates, depending, for example, on the type of feedstock and handling or storage of the feedstock prior to entering the extraction process. With this embodiment, the liquid does not have to be completely removed from the solids prior to the hot water co-extraction process. In either case, the solids are eventually separated from the liquid stream generated by the physical treatment and/or hot water extraction steps. This liquid stream and/or solids stream may be further processed to bio-products according to conventional methods known in the art. (See, Biomass, Top value Added Chemicals from Biomass; U.S. Dept. of Energy, August 2004; Biotechnological Routes to Biomass Conversion; Nat'l Renewable Energy Lab., August 2004)

In a further embodiment of this aspect, the pH of the process may be adjusted prior to heating to between approximately 3 and 8, preferably between approximately 4 and 7, and more preferably between approximately 5 and 6.

In yet another embodiment of this aspect, the water added to the feedstock may be pretreated to the desired temperature.

In a further embodiment of this aspect, water is added to the feedstock in a cook vessel, as above, and the extraction process proceeds for approximately 40-60 minutes at the lower end of range of temperatures employed in the inventive method, for example 120° C. to 150° C. The liquid in the vessel is then removed and retained and a second volume of water is added to the vessel containing the partially extracted feedstock solids. The extraction process continues for an additional approximately 60 minutes at 150° C. to 180° C. The liquid in the vessel is then removed and combined with the first liquid from the lower temperature extraction.

In a third aspect, the liquid stream of the method of the first aspect above can be purified to produce sugar or molasses or bioactive compounds, or further processed, for example, fermented or catalyzed into ethanol, other alcohols, alkanes or other bio-products by means known in the art. In one embodiment of this aspect, the liquid stream can be combined with soluble sugars that have been separately produced by other processes known in the art for fermentation to ethanol or other bio-products. In another embodiment of this aspect, the solubilized carbohydrates and bioactive compounds may be isolated, optionally concentrated and/or purified, and used separately to produce high-value bio-products.

In a fourth aspect, the invention provides compositions, such as, a liquid stream of fructose, glucose, sucrose and other soluble oligosaccharides from hemicellulose and soluble bioactive compounds produced by the method of the present invention.

In a fifth aspect, the method of the invention provides the means to generate compositions derived from further processing of the liquid stream, such as sugars, biofuels and other bio-products. The liquid stream can also be utilized in other processes, such as in production of algal lipids, butanol, alkanes, citric acid, and succinic acid. The liquid stream can also be further processed to purify and/or concentrate bioactive compounds such as gluco- and xylo-oligosaccharides, tannins and polyphenols or other extractives that may have health benefits.

In a sixth aspect, the solids stream of the method of the first aspect above can be further processed, for example, by activation of cellulose followed by an enzymatic or acid catalyzed hydrolysis into sugars that may be used to produce bio-alcohols or other bio-products.

In a seventh aspect, the invention provides compositions, such as, a solids stream of cellulose and lignin produced by the method of the invention.

In an eighth aspect, the method of the invention provides the means to generate compositions derived from further processing of the solids stream.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### DETAILED DESCRIPTION OF PREFERRED EMBODIMENT

The present invention generally relates to the extraction of sucrose, xylo-oligosaccharides, xylose, bioactive compounds and other lignin-derived compounds from feedstock and, in particular, to hot water co-extraction of sucrose (fructose and glucose), xylo-oligosaccharides, xylose, extractives, tannins, polyphenols and other related compounds solubilized and derived from lignin.

The discoveries described herein provide a means to greatly simplify and reduce the cost for production of sugars for fermentation to alcohols or other uses, from feedstocks that include sucrose, fructose, glucose and/or hemicellulosic sugars. In contrast to conventional milling and extraction of sucrose, accompanied by a separate second generation process focused on treatment of bagasse to produce biomass-derived C5 sugars, the novel method described herein allows co-extraction of sucrose and hemicellulosic sugars in a hot-water extraction process with minimal production of inhibitors. In consequence, practice of the invention simplifies the process and provides a reduction in capital and operating costs by permitting co-extraction in a single cook vessel, while yielding a mixture of soluble sugars accompanied by extractives, phenolics and other bioactive compounds, while limiting production of inhibitors of fermentation.

Discovery of the extraction process of the invention has led to studies performed with intact and milled energy cane bagasse, and also with raw sucrose as control, at elevated temperatures and with retention times up to 4 hours. Discovery of the inventive process has made possible an extraction process, that produces minimal degradation of sugars, with greater than 95% preservation of the total sugar content of the cane, when performed at the lower temperatures within the inventive range, such as at 130° to 150° C. At temperatures above 160° C., some sugar degradation is noted after extended retention times. Additionally, it has been discovered that the C6 sugars in these extracts are fermentable, with no adverse effects on yield or rate of ethanol production.

The lignocellulosic feedstock, whether in tact or used as bagasse, can be physically processed to provide particulates of increased surface area. Processed fractions typically have a longest dimension of less than about 10 cm, and can include fines whose longest dimensions are less than about 1 mm.

In summary, it has been discovered that by use of the method of the invention, as described herein, it is possible to extract and control degradation of sucrose in conjunction with extraction and solubilization of hemicellulose sugars and other biomass-derived compounds, in a single cook vessel and process. Specifically, results reported herein indicate that it is feasible to extract sucrose and xylo-oligosaccharides at temperatures ranging between 120° C. and 180° C., while preserving a significant percentage of sugars that are fermentable that are not degraded by the inventive hot-water extraction process.

### EXAMPLES

The following examples have been provided as guidance to one of ordinary skill in the art in practicing several embodiments of the invention. These examples are exemplary only and one skilled in the art would appreciate that changes, modifications and alterations can be employed without departing from the scope of the subject matter disclosed herein.

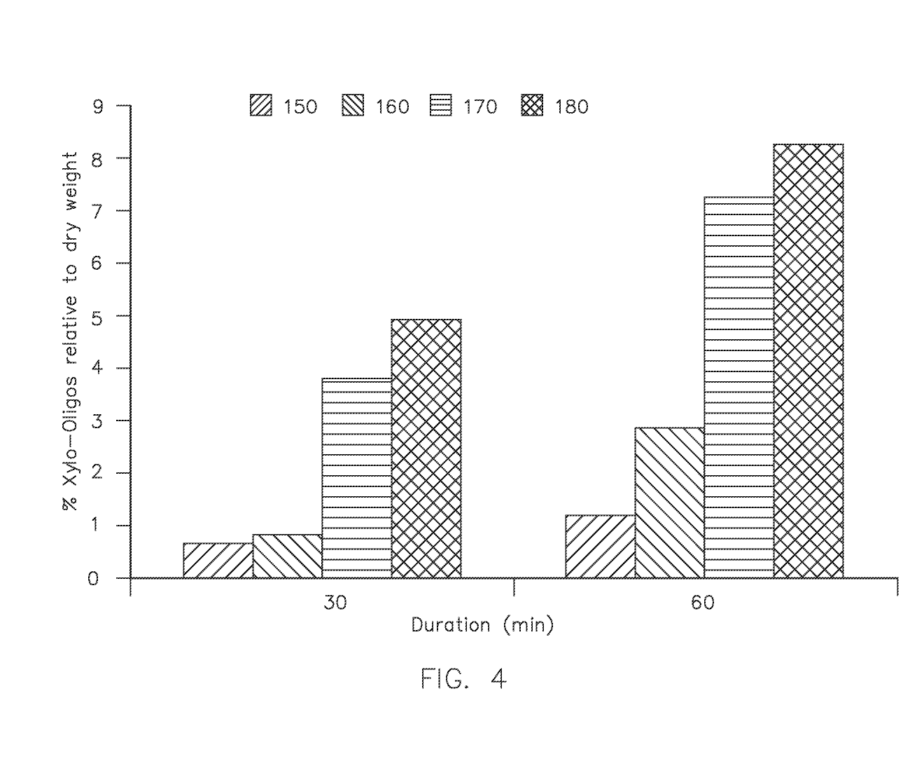

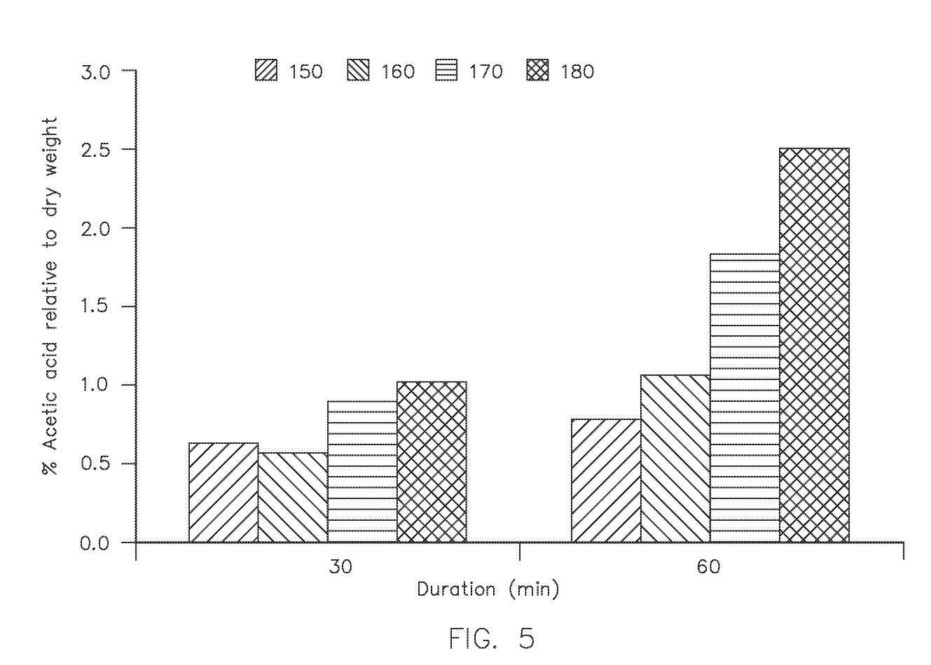

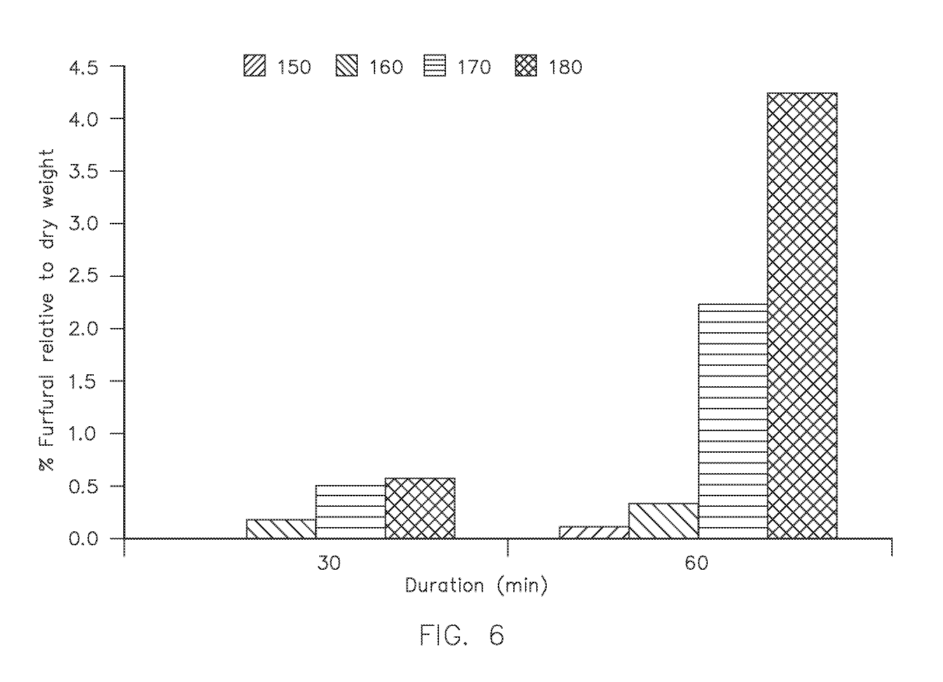

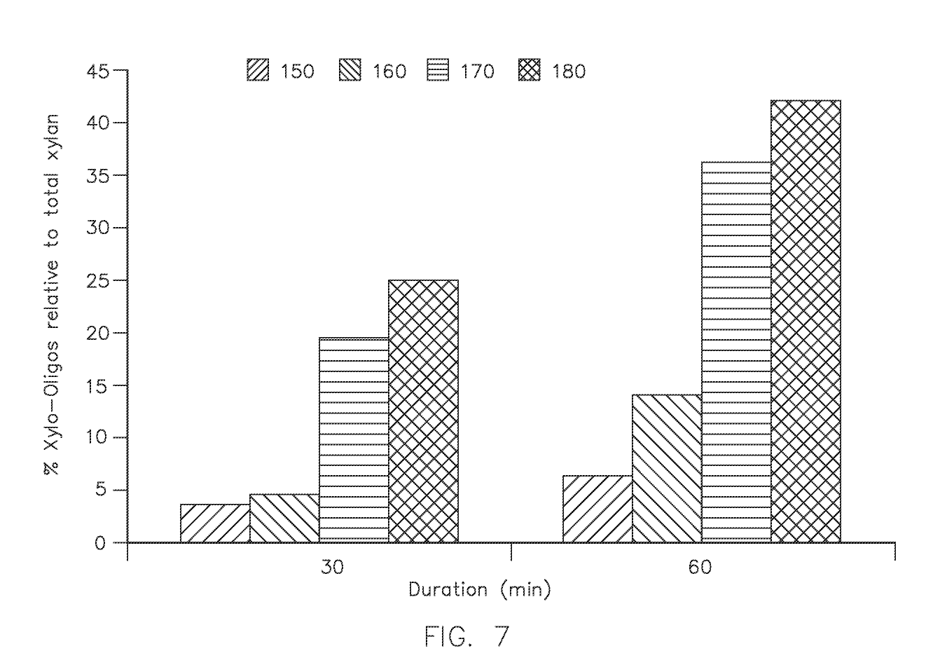

FIGS. 4-7 illustrate the impacts of process conditions upon the extraction of xylo-oligosaccharides, the release of acetic acid and the production of furfural. In these figures, we compare the results from examples 9 and 11 with results obtained from similar experiments at 150° C. and 180° C. (not otherwise shown). FIG. 4 (% Xylo-oligos relative to dry weight) illustrates that higher temperatures lead to greater amounts of xylo-oligosaccharides in the extract, and also illustrate that increasing the extraction time from 30 to 60 minutes can significantly enhance xylo-oligosaccharide production. FIG. 7 (xylo-oligosaccharides relative to total xylan content). FIG. 5 (Acetic acid relative to dry weight) also illustrates greater amounts of acetic acid detected in the extract when the process is conducted at higher temperatures and/or over a longer duration. FIG. 6 (furfural production relative to dry weight) illustrate the significant change in furfural generation at incubation time and temperature are increased. Comparing the results from FIG. 6 with those from FIGS. 4 and 5, it is apparent that, compared to xylo-oligosaccharide production, furfural production is much more sensitive to incubation time and temperature. Thus, there is an upper limit on incubation time and temperature that may be employed, if the objective is to limit the production of furfural and similar inhibitors. Conversely, if the goal is to produce furfural, then higher temperatures and incubation times may be employed, ultimately converting the carbohydrates into furfural and HMF. It is important to note that longer incubation times are feasible at lower temperatures, whereas shorter incubation times are preferred at higher temperatures.

### Example 1

### Sugar Extraction from Milled Energy Cane

Raw energy cane (stalks only) was milled using a knife mill, the juice was removed and the collected wet solids were used in a trial of sugar extraction. For each trial condition, four individual fiber samples were prepared, ranging in mass from 20 to 40 g (wet basis; estimated moisture content approximately 65%). Water was added to each sample vessel, in an amount approximately double the wet mass of the energy cane sample. The sample vessels were sealed and the contents cooked in an autoclave at 125° C. for 1, 2, 3, or 4 hours. For each temperature/time combination, the four individual samples were pooled after cooking. The pH of the liquid extract was measured and compared with the initial pH of 6.2, and the sugar and inhibitor content of the liquid extract was measured using HPLC, on an Agilent 1200 series HPLC system with a Biorad carbohydrate column and a Biorad organic acids column. A set of standards was run co-currently, as a benchmark of sugar recovery and degradation. Concentrations of sucrose, glucose, xylose, fructose, acetic acid, formic acid, furfural and 5-HMF were determined (not separately reported).

Key results are summarized in Table 1. None of the liquid samples contained xylose. Consequently, the percentage extracted represents the aggregate amount of sucrose, glucose and fructose measured in the liquid samples. Extraction percentages are expressed in terms of total mass of wet sample. The percentage of sugar extracted from milled energy cane increased with incubation time. The pH of the liquid extract decreased with incubation time, from 5.0 to 4.4, compared to the original pH of the liquid (6.2), coinciding with formation of organic acids as degradation products, and extraction of acetic acid originally present as acetyl-xylan in the energy cane fiber fraction. The percentage of sugars converted into degradation products such as HMF, furfural and formic acid increased slightly with incubation time; nonetheless, only about 1% of the total sugars extracted was converted into degradation products.

TABLE 1
Sugar extraction from energy cane at 125° C.

| cook time | milled samples | | | | billets | | | | sucrose | |
|---|---|---|---|---|---|---|---|---|---|---|
| in hours at | | % sugars | % | % degradation | | % sugars | % | % degradation | % | % lost or |
| 125° | pH | extracted | acetate | products | pH | extracted | acetate | products | hydrolyzed | degraded |
| 1 | 5.0 | 1.4 | 8.1 | 0.7 | 5.1 | 1.2 | 0.8 | 0.6 | 11% | 0 |
| 2 | 4.9 | 1.7 | 14.8 | 0.8 | 4.8 | 2.2 | 0.9 | 0.4 | 66% | 0 |
| 3 | 4.7 | 2.7 | 14.0 | 1.1 | 4.7 | 3.2 | 0.9 | 0.5 | 58% | 0 |
| 4 | 4.4 | 3.0 | 15.6 | 1.1 | 4.8 | 2.8 | 1.4 | 0.6 | 95% | 3 |

### Example 2

### Sugar Extraction from Cut Energy Cane Billets

Raw energy cane was cut into lengths, and the collected billets were used in a trial of sugar extraction. For each trial condition, four individual fiber samples were prepared, ranging in mass from 15 to 23 g (wet basis; estimated moisture content approximately 65%). Water was added to each sample vessel, in an amount approximately double the wet mass of the energy cane sample. The sample vessels were sealed and the contents cooked in an autoclave at 125° C. for 1, 2, 3, or 4 hours. For each temperature/time combination, the four individual samples were pooled after cooking. The pH of the liquid extract was measured and compared with the initial pH, and the sugar and inhibitor content of the liquid extract was measured using HPLC, on an Agilent 1200 series HPLC system with a Biorad carbohydrate column and a Biorad organic acids column. A set of standards was run co-currently, as a benchmark of sugar recovery and degradation. Concentrations of sucrose, glucose, xylose, fructose, acetic acid, formic acid, furfural and HMF were determined.

Key results are summarized in Table 1. None of the liquid samples contained xylose. Consequently, the percentage sugars extracted represents the aggregate amount of sucrose, glucose and fructose measured in the liquid samples. Sugar extraction percentages are expressed in terms of total mass of wet sample. The percentage of sugar extracted from energy cane billets generally increased with incubation time, although a reduction was noted between the 3 hour and 4 hour incubation samples, which may not be statistically significant. The pH of the liquid extract decreased with incubation time, from 5.1 to 4.8, compared to the original pH of the liquid (6.2), coinciding with production of organic acids from sugar degradation and extraction of acetic acid originally present as acetyl-xylan in the energy cane fiber fraction. The percentage of sugars converted into degradation products such as HMF, furfural and formic acid did not measurably change with incubation time; nonetheless, only about 0.5% of the total sugars extracted was converted into degradation products

### Example 3

### Control—Hydrolysis and Degradation of Raw Sucrose Juice

10 wt % sucrose solutions were prepared by dissolving 1.2 to 1.7 g of crystalline sucrose in water. The resulting solutions were transferred into sample cook vessels and sealed, then cooked in an autoclave at 125° C. for 1, 2, 3, or 4 hours. The sugar content and inhibitors content of the liquid extract was measured using HPLC, on an Agilent 1200 series HPLC system with a Biorad carbohydrate column, and a Biorad acids column. A set of standards was run co-currently, as a benchmark of sugar recovery and degradation. Concentrations of sucrose, glucose, xylose, fructose, acetic acid, formic acid, furfural and HMF were determined.

Key results are summarized in Table 1. The percentage sugars extracted represents the aggregate amount of sucrose, glucose and fructose measured in the liquid samples. At incubation times from 1 to 3 hours, total sugars recovered matched the initial total mass of sucrose in solution. However, after 4 hours of incubation at 125° C., 3% of the sucrose was unaccounted for. The percentage of the original sucrose hydrolyzed into glucose and fructose tended to increase with incubation time, from 11% after 1 hour, to 95% after 4 hours.

### Example 4

### Sugar Extraction from Milled Energy Cane

Raw energy cane was milled using a knife mill, and the collected solids were used in a trial of sugar extraction. For each trial condition, four individual fiber samples were prepared, ranging in mass from 13 to 16 g (wet basis; estimated moisture content approximately 65%). Water was added to each sample vessel, in an amount approximately double the wet mass of the energy cane sample. The sample vessels were sealed and the contents cooked in an autoclave at 130° C. for 1 hour. After cooking, the four individual samples were pooled. The pH of the liquid extract was measured and compared with the initial pH, and the sugar and inhibitor content of the liquid extract was measured using HPLC, on an Agilent 1200 series HPLC system with a Biorad carbohydrate column and a Biorad organic acids column. A set of standards was run co-currently, as a benchmark of sugar recovery and degradation. Concentrations of sucrose, glucose, xylose, fructose, acetic acid, formic acid, furfural and HMF were determined.

Key results are summarized in Table 2. None of the liquid samples contained xylose. Consequently, the percentage sugars extracted represents the aggregate amount of sucrose, glucose and fructose measured in the liquid samples. Extraction percentages are expressed in terms of total mass of wet sample. 2.5 wt % of the wet energy cane sample was recovered as sucrose, glucose and fructose. The pH of the liquid extract decreased to 4.7 compared to the original pH of the liquid (6.2), coinciding with formation of organic acids as degradation products, and extraction of acetic acid originally present as acetyl-xylan in the energy cane fiber fraction. The percentage of sugars converted into degradation products such as HMF, furfural and formic acid was only about 0.1% of the total sugars extracted.

TABLE 2
Sugar Extraction from energy cane at 130° C.

| incubation | milled samples | | | | billets | | | | sucrose | |
|---|---|---|---|---|---|---|---|---|---|---|
| time in | | % sugars | % | % degradation | | % sugars | % | % degradation | % | % lost or |
| hours | pH | extracted | acetate | products | pH | extracted | acetate | products | hydrolyzed | degraded |
| 1 | 4.7 | 2.5 | 5.8 | 0.1 | 5.0 | 0.8 | 1.5 | 0.2 | 11% | 0 |

### Example 5

### Sugar Extraction from Cut Energy Cane Billets

Raw energy cane was cut into lengths, and the collected billets were used in a trial of sugar extraction. Four individual fiber samples were prepared, ranging in mass from 30 to 35 g (wet basis; estimated moisture content approximately 65%). Water was added to each sample cook vessel, in an amount approximately double the wet mass of the energy cane sample. The sample vessels were sealed and the contents cooked in an autoclave at 130° C. for 1 hour. After cooking, the four individual samples were pooled. The pH of the pooled liquid extract was measured and compared with the initial pH of 6.2, and the sugar and inhibitor content of the liquid extract was measured using HPLC, on an Agilent 1200 series HPLC system with a Biorad carbohydrate column and a Biorad organic acids column. A set of standards was run co-currently, as a benchmark of sugar recovery and degradation. Concentrations of sucrose, glucose, xylose, fructose, acetic acid, formic acid, furfural and HMF were determined.

Key results are summarized in Table 2. None of the liquid samples contained xylose. Consequently, the percentage sugars extracted represents the aggregate amount of sucrose, glucose and fructose measured in the liquid samples. Extraction percentages are expressed in terms of total mass of wet sample. 0.8 wt % of the wet energy cane sample was recovered as sucrose, glucose and fructose. The pH of the liquid extract decreased to 5.0 compared to the original pH of the liquid (6.2), coinciding with formation of organic acids as degradation products, and extraction of acetic acid originally present as acetyl-xylan in the energy cane fiber fraction. The percentage of sugars converted into degradation products such as HMF, furfural and formic acid was only about 0.2% of the total sugars extracted.

### Example 6

### Control—Hydrolysis and Degradation of Sucrose Solution

A 10 wt % sucrose solution was prepared by dissolving 1.15 g of crystalline sucrose in water. The resulting solution was transferred into a sample cook vessel and sealed, then cooked in an autoclave at 130° C. for 1 hour. The sugar content and inhibitors content of the liquid extract was measured using HPLC, on an Agilent 1200 series HPLC system with a Biorad carbohydrate column, and a Biorad acids column. A set of standards was run co-currently, as a benchmark of sugar recovery and degradation. Concentrations of sucrose, glucose, xylose, fructose, acetic acid, formic acid, furfural and HMF were determined.

Key results are summarized in Table 2. The percentage sugars extracted represents the aggregate amount of sucrose, glucose and fructose measured in the liquid samples. After 1 hour of incubation at 130° C., total sugars recovered matched the initial total mass of sucrose in solution, indicating no degradation. Only 11% of the original sucrose was hydrolyzed into monomers (fructose and glucose).

### Example 7

### Control Comparison—Hydrolysis and Degradation of Sucrose Solution Under Acidic Conditions

10 wt % sucrose solutions were prepared by dissolving crystalline sucrose in a solution of acetic acid and water. The amount of acetic acid ranged from 0.1 to 2 vol %. The resulting solutions were transferred into sample cook vessels and sealed, then cooked in an autoclave at 130° C. for 1 hour. The sugar content and inhibitors content of the liquid extract was measured using HPLC, on an Agilent 1200 series HPLC system with a Biorad carbohydrate column, and a Biorad acids column. A set of standards was run co-currently, as a benchmark of sugar recovery and degradation. Concentrations of sucrose, glucose, xylose, fructose, acetic acid, formic acid, furfural and HMF were determined (not separately reported).

Key results are summarized in Table 3. Under acidic conditions, 100% of the sucrose was hydrolyzed to glucose and fructose after 1 hour of incubation at 130° C., compared to only 11% hydrolysis at 130° C. when acid was not added (Example 6). Furthermore, the percent degradation increased as the acidity of the solution increased. However, even with 20 g/L acetic acid in the solution, only 1.2% of the sugars were degraded into furfural, HMF, and formic acid after 1 hour of incubation at 130° C. Thus, even with significant acid present, under these conditions, there is negligible degradation of sucrose, glucose, and fructose.

TABLE 3
sucrose degradation under acidic conditions

| vol % | % sucrose | % degradation |
|---|---|---|
| acetic acid | hydrolyzed | products |
| 0.1 | 100% | 0.3 |
| 0.5 | 100% | 0.6 |
| 1 | 100% | 0.9 |
| 2 | 100% | 1.2 |

### Example 8

### Sugar Extraction from Milled Energy Cane without Imbibition

Raw energy cane was milled using a 2 ton/d sugar cane mill/press system, operating either with or without imbibition water. Samples of the milled/extracted solids were collected and used in a trial of sugar extraction. Samples 1 and 2 comprised samples that were milled and pressed, but did not include imbibition water in the process. Samples 3 and 4 were obtained from a milling process that included imbibition water and the resulting solids thus possessed less residual sucrose than samples 1 and 2. A control sample (#5) was prepared using 1 g of sucrose and 10 g of water.

Extraction samples were prepared using 16 to 19 g of milled energy cane (wet basis; average moisture content approximately 65%). Water was added to each sample cook vessel, in an amount approximately equal to 4 times the wet mass of the energy cane sample. The five sample vessels were sealed and the contents cooked in an autoclave at 130° C. for 2.5 hours. After cooking, the sugar and inhibitor content of the liquid extract was measured using HPLC, on an Agilent 1200 series HPLC system with a Biorad carbohydrate column and a Biorad organic acids column. A set of standards was run co-currently, as a benchmark of sugar recovery and degradation. Concentrations of sucrose, glucose, xylose, fructose, acetic acid, formic acid, furfural and HMF were determined. A compositional analysis was performed on the original biomass sample to determine the glucan and xylan content of the fiber fraction before extraction. Similarly, a compositional analysis was performed on the liquid extract to determine the amount of gluco-oligosaccharides (from cellulose) and xylo-oligosaccharides (from hemicellulose) in the extract.

Glucose and fructose were the primary carbohydrate monomers recovered, with trace amounts of dimers, sucrose and xylose. In the control, 100% of the sucrose was hydrolyzed, and 97% of the original sucrose was recovered, implying approximately 3% was unaccounted for or degraded. Approximately 0.15% of the initial sucrose (lg.) was recovered as HMF.

Furfural was not detected in any of the samples, and HMF was detected in only one of the four fiber samples processed, representing less than 0.01% of the initial wet cane sample. Acetic acid was detected in the extract from all four fiber samples, representing 2 to 4% of the dry fiber mass. Acetic acid is released during the extraction of xylan and production of soluble xylo-oligosaccharides and xylose.

The extracts from the pressed fiber and the fiber pressed with imbibition water contained 4.5 and 2.8 g/L of gluco-oligosaccharides, respectively, compared to 1.8 g/L and 0.55 g/L of free glucose, respectively, primarily from extracted sucrose. The extracts from both the pressed fiber and the fiber pressed with imbibition water each contained 3.0 g/L of xylo-oligosaccharides. These results indicate that under either of these conditions, about 20% of the xylan in the fiber can be co-extracted with sucrose, with minimal degradation of the extracted sugars.

Examples 9-11 and 13 used a dual-vessel MK Digester from MK Systems, Inc. (Peabody, Mass.), comprising two 10 L cook vessels capable of operating at temperatures up to 225° C. The system contains band heaters and immersion heaters for rapid heating of the vessels and temperature control. In the studies cited below, one vessel was used to preheat water, which, after reaching the target cook temperature, was transferred to the second vessel that contained the biomass, where the cooking/digestion takes place (cook vessel). In the designated examples below, we refer to these two vessels are referred to as the “pre-heat vessel” and “cook vessel”, respectively. The vessels contain an external pump-loop that allows the solvent/extract to be recirculated and distributed across the static bed of biomass present at the bottom of the cook vessel. The recirculation loop contains sampling lines that allow samples to be safely collected from the recirculation loop while maintaining pressure and temperature during the digestion process. During operation, the cook vessel will contain solid biomass, water/extract in liquid form, and vapor generated by evaporation of the solvent. The operating pressure of the digester cook vessel corresponds to the saturation vapor pressure of the solvent at the specified operating temperature for the experiment.

### Example 9

### Co-Extraction of Sucrose and Xylo-Oligosaccharides from Milled Energy Cane

Energy cane was milled, creating irregular shapes of up to approximately 10 cm in length, and 283 g (dry basis) cane was added to the cook vessel. Five kg of water preheated in the “pre-heat vessel” was transferred to the digester “cook vessel” and the contents were cooked at 160° C. for 90 minutes. Samples were collected at regular intervals over the entire duration of the extraction process, and the entire volume of extraction liquid was drained from the cook vessel after 90 minutes of incubation. The content of carbohydrates (monomers and oligomers), organic acids and inhibitors, and total solubles (including lignin-derived compounds) was determined for each sample. The total mass of extract collected was determined, along with the mass of wet energy cane. The production of sugar monomers, sugar oligomers, organic acids and inhibitors, and total solubles was thus determined as a function of extraction time.

The extract collected after 90 minutes of cooking/extraction contained 77 g of total solids, including 14.8 g of xylo-oligosaccharides, 10.1 g of inhibitors (including 3.9 g of acetic acid and 2.0 g of furfural), and 12.1 g of sucrose or its monomer dissociation products. By comparison, after 60 minutes, the extract contained 68 g of total solids, 7.6 g of xylo-oligosaccharides, but only 7.0 g of inhibitors (including 1.0 g of furfural), with more sucrose/glucose/fructose. The additional incubation time led to increased extraction of xylo-oligosaccharides, but also increased production of furfural as sucrose-derived compounds were degraded. However, the total inhibitors produced were less at 160° C. compared to 170° C. (Example 11)

The presence of lignin-derived compounds such as polyphenols, flavonoids and derivatives of benzoic acid was evaluated by measuring the absorbance of the extracts at 280 nm, an absorbance range where these compounds are known to be detectable (Gorinstein et al., Spectroscopic Analysis of Polyphenols in White Wines, J Fermentation and Bioengineering, 75(2), 115-120 (1993)). The intensity of the absorbance measurement was so high that samples had to be diluted with water (up to a 500-fold dilution) before the absorbance measurement could be completed. After correcting for sample dilution, a relative absorbance number (RAN) was assigned, equal to the product of the absorbance and the dilution factor. In these experiments at 160° C., the RAN increased from 96 after 30 minutes of incubation, to 141 after 60 minutes, and 192 after 90 minutes. The increase in RAN coincided with an increase in the color intensity of the extract that would be consistent with increased production of lignin-derived compounds.

### Example 10

### Co-Extraction of Sucrose and Xylo-Oligosaccharides from Bagasse

Energy cane was processed through a juicing system to remove most sucrose, and the bagasse (with some residual sucrose) was then milled. The energy cane bagasse (149 g dry basis) was added to the digester cook vessel. Preheated water (2.39 kg) was transferred from the preheat vessel to the digester cook vessel and the bagasse was cooked at 150° C. for 90 minutes. Samples were collected from the cook vessel at regular intervals over the entire duration of the extraction process, and the entire volume of extraction liquid was drained from the cook vessel after 90 minutes of incubation. The content of carbohydrates (monomers and oligomers), organic acids and inhibitors, and total solubles (including lignin-derived compounds) was determined for each sample. The total mass of extract collected was determined, along with the mass of wet energy cane. The production of sugar monomers, sugar oligomers, organic acids and inhibitors, and total solubles was thus determined as a function of extraction time.

The extract collected after 90 minutes of cooking/extraction contained 23.6 g of total solids, including 3.0 g of xylo-oligosaccharides, 2.0 g of inhibitors (including 1.5 g of acetic acid and no furfural), and 2.1 g of sucrose or its monomer dissociation products. By comparison, after 60 minutes, the extract contained 17.2 g of total solids, but only 1.2 g of inhibitors and 0.9 g of acetic acid, and essentially the same amount sucrose/glucose/fructose. Under these conditions, it is possible to co-extract xylo-oligosaccharides and sucrose from bagasse while producing negligible amounts of furfural.

### Example 11

### Co-Extraction of Sucrose and Xylo-Oligosaccharides from Milled Energy Cane

Energy cane was milled, creating irregular shapes of up to approximately 10 cm in length, and 282 g (dry basis) cane was added to the cook vessel. Five kg of preheated water was transferred from the preheat vessel to the digester cook vessel and the contents were cooked at 170° C. for 60 minutes. Samples were collected from the cook vessel at regular intervals over the entire duration of the extraction process, and the entire volume of extraction liquid was drained from the cook vessel after 60 minutes of incubation. The content of carbohydrates (monomers and oligomers), organic acids and inhibitors, and total solubles (including lignin-derived compounds) was determined for each sample. The total mass of extract collected was determined, along with the mass of wet energy cane. The production of sugar monomers, sugar oligomers, organic acids and inhibitors, and total solubles was thus determined as a function of extraction time.

The extract collected after 60 minutes of cooking/extraction contained 72.8 g of total solids, including 20 g of xylo-oligosaccharides, 19 g of inhibitors (including 5.1 g of acetic acid and 6.5 g of furfural), and 10.3 g of sucrose or its monomer dissociation products. By comparison, after 30 minutes, the extract contained 70.8 g of total solids, but only 8.4 g of inhibitors and 1.5 g of furfural, with more sucrose/glucose/fructose. The additional incubation time led to increased extraction of xylo-oligosaccharides, but also additional production of furfural.

The presence of lignin-derived compounds such as polyphenols, flavonoids and derivatives of benzoic acid in the extracts was evaluated by measuring the absorbance of the extracts at 280 nm. After correcting for sample dilution, a relative absorbance number (RAN) was assigned, equal to the product of the absorbance and the dilution factor. In these experiments at 170° C., the RAN increased from 201 after 30 minutes of incubation, to 354 after 60 minutes. The increase in RAN coincided with an increase in the color intensity of the extract that would be consistent with increased production of lignin-derived compounds.

### Example 12

### Co-Extraction from Crushed Energy Cane at 130° to 170° C. Up to 8 Hours

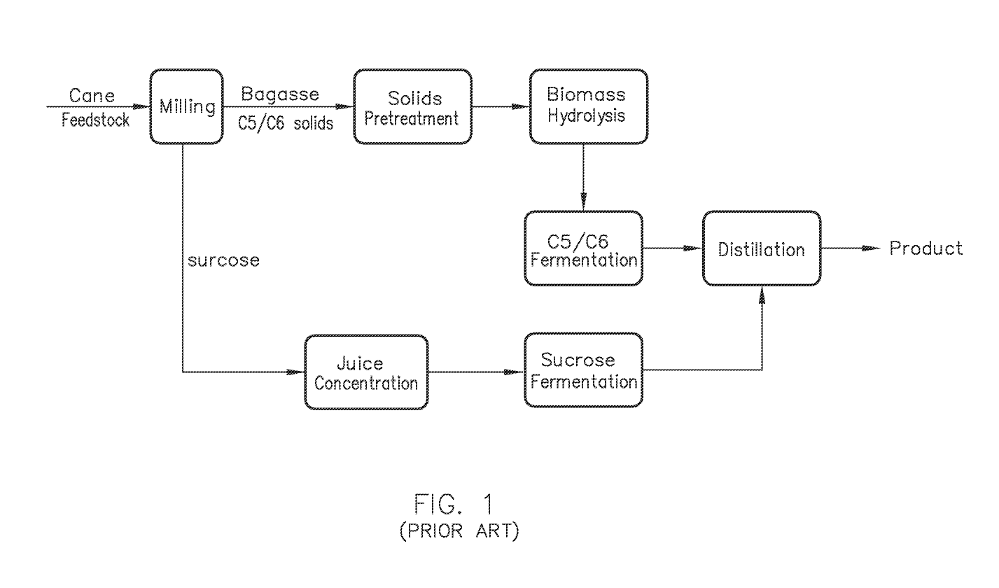

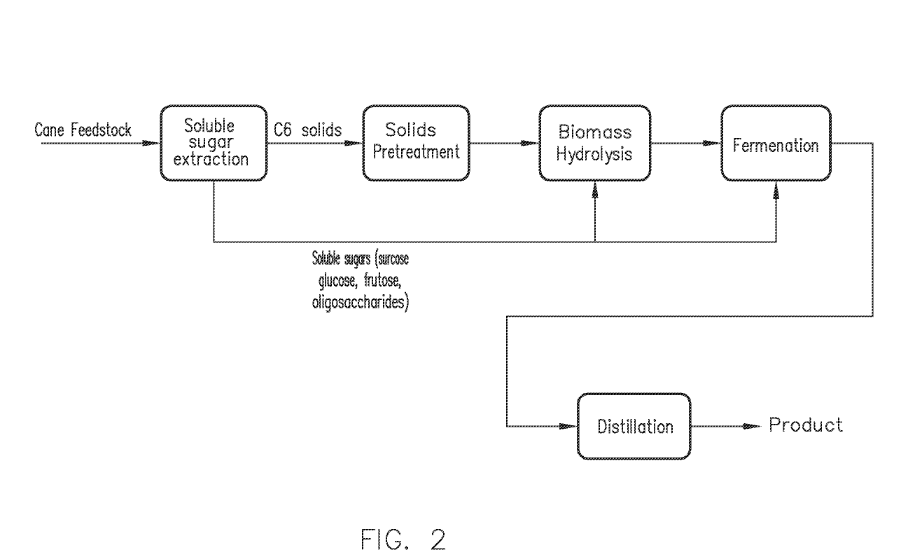

The inventive, simplified process for extraction of sugars and production of biochemicals from sucrose-rich feedstocks is illustrated in FIGS. 2 and 3. Harvested feedstock was used intact (FIG. 2) or was first milled (FIG. 3). The intact or milled feedstock was then fed to a cooking system in a cook vessel, and combined with water in sufficient volume to keep the water in the liquid state during subsequent heating, followed by cooking/digestion in the same cook vessel under pressure at a range of 130° to 170° C., for from 30 minutes to 8 hours to co-extract sucrose, glucose, fructose, xylose, xylo-oligosaccharides, extractives, bioactive compounds, and soluble lignin-derived compounds such as tannins and polyphenols. The resulting liquid and solids fractions are separated to yield a liquid stream and a solids stream. If it is desired to further process the solids stream by pretreatment and activation, the separation may be done while the pressure is retained. In contrast, if it is desired to recover alpha cellulose from the solids stream, the separation may be done under near ambient conditions. The resultant liquid stream may be directed to either a fermentation system, or to a hydrolysis reactor for further breakdown of oligosaccharides. The liquid is removed, for example, without limitation, by pressing, filtration, or other system known in the art, leaving a solids fraction rich in cellulose and lignin, but reduced in hemicellulose. Pressing may be done under pressure, whereas filtration may be done under either pressure or a vacuum. The solids fraction can be sent to a biomass pretreatment system, where the lignin can be disrupted and the cellulose can be activated at high temperatures and pressures, according to methods well known in the art. Such methods include, without limitation, autohydrolysis, steam explosion, sulfuric acid or SO₂-catalyzed steam explosion, ammonia fiber expansion, and alkaline pretreatment, among others. The resulting activated cellulose fraction can then be hydrolyzed using enzymes (cellulases, accessory enzymes, etc.) to produce glucose. Optionally, the hydrolysis reactor can also include the liquid stream removed from the inventive cooking process (above). The resulting sugar product from the hydrolysis reactor may be used in raw form, purified, or further processed, e.g., by fermentation, as shown in FIG. 1. The fermentation products may be purified, for example, by distillation, to recover the desired bio-fuel or other bio-product.

### Example 13

### Dual Solvent Co-Extraction of Sucrose and Xylo-Oligosaccharides from Crushed Energy Cane

In this example, we illustrate the selective extraction of sucrose and sucrose-derived monomers and xylo-oligosaccharides, while employing two solvent addition steps.

Energy cane (0.21 kg dry basis) was crushed and placed in a biomass digester cook vessel, to which 2.5 kg of water was added to create a slurry. The contents were heated and held at 130° C. over a total of 43 minutes, and then the liquid (including solubilized components) was drained from the cook vessel. 2.4 kg of fresh water (preheated to 180° C. in the preheat vessel) was added to the cook vessel, and the biomass digestion continued for another 60 minutes at 180° C. The preheating of fresh water is not required, though desirable for increased efficiency. Samples were collected from the cook vessel at regular intervals over the entire duration of the extraction process. The content of carbohydrates (monomers and oligomers), organic acids and inhibitors, and total solubles (including lignin-derived compounds) was determined for each sample. The total mass of extract collected from both cook steps was determined, along with the mass of wet energy cane remaining at the conclusion of the digestion process. The production of sugar monomers, sugar oligomers, organic acids and inhibitors, and total solubles was thus determined as a function of extraction time.

During the 130° C. extraction, 15 g of sucrose (or its monomers) was recovered, along with 0.7 g of xylo-oligosaccharides, 0.2 g of acetic acid, and 0.7 g of total inhibitors. The total mass of solubles recovered, including lignin-derived phenolics and extractives, was 30.6 g. Extraction was continued at 180° C. and additional 3.3 g of sucrose (or its corresponding monomers) was recovered, along with 25.9 g of xylo-oligosaccharides, 4.5 g of acetic acid, and 15.9 g of total inhibitors (which includes acetic acid; some inhibitors are produced from extracted carbohydrates). The incremental amount of dissolved solids extracted is 52.8 g. It is apparent from this example that this dual solvent extraction process can preferentially extract sucrose at a temperature from approximately 120° C. to approximatley 150° C. within the lower range of the inventive method, and xylo-oligosaccharides in the second solvent at a temperature of approximately 150° to approximately 180° C. within the upper range of the invention. This may pose an advantage if the objective is to generate separate, purified sugars rather than a mixed sugar stream. Both streams may also contain biomass extractives, lignin-derived compounds such as polyphenols, and/or other bioactive compounds.

### Example 14

### Uses for Xylo-Oligosaccharides Extracted in the Method of the Invention

The extracts isolated from these sucrose-laden feedstocks (Examples 1, 2, 5 and 8-13) may also be rich in compounds that have health benefits, or which serve as precursors for production of biochemicals or biopolymers. For example, the extracts contain xylo-oligosaccharides that can function as prebiotics, when used either as is, after purification, after enzymatic hydrolysis or modification, or after derivatization (Aachary and Prapulla, “Xylo-oligosaccharides (XOS) as an emerging prebiotic: Microbial Synthesis, Utilization, Structural Characterization, Bioactive Properties, and Applications”, Comprehensive Reviews an Food Science and Food Safety, 10(1), 2-16 2011). These xylo-oligosaccharides can also be fully hydrolyzed chemically or enzymatically to produce xylose, which can be converted catalytically to furfural, or used as a precursor for polyol production, such as xylitol, which is a high value alternative sweetener.

The extracts produced via the claimed processes (obtained in Examples 1, 2, 5 and 8-13) contain xylo-oligosaccharides, and these crude extracts can be processed by methods known in the art, such as chromatographic separation, membrane filtration, and ultrafiltration, to isolate the xylo-oligosaccharide fractions. In the case of membrane processes, the choice of molecular weight cut-off for the membrane (e.g., lkDa, 3 kDa, 5 kDa, or 10 kDa) can be used to isolate oligosaccharides with different degrees of polymerization, if desired.

Alternatively, the xylo-oligosaccharides from the Examples may be hydrolyzed with dilute acids or with xylanases that contain endo- and/or exo-xylanases and/or xylobiohydrolase to break down the soluble oligosaccharides into xylose monomer. The xylose monomers can be isolated from unconverted oligomers and from other compounds in the extract, such as organic acids and lignin-derived compounds via chromatographic separation, such as is practiced industrially for a range of carbohydrates. The resulting purified xylose fraction can be concentrated, used as is, or used to produce, for example, xylitol via catalytic or fermentation methods. The conditions for each of these process steps can be optimized by one skilled in the art, taking into account the degree of polymerization of the xylo-oligosaccharides, the type of enzyme or dilute acid, the types and amounts of other impurities present in the extracts, the type/size of resin used for purification, the hydrogenation catalyst, or the type of fermentation organism.

### Example 15

### Uses for Lignin-Derived Extracts

The extracts described in examples 1, 2, 5 and 8-13 also contain compounds that are derived from lignin, which is naturally present in lignocellulosic biomass. Lignin exists in several forms, including insoluble fractions and fractions that are soluble and extractable under acidic, neutral or alkaline conditions. Soluble lignin-derived phenolics can include ferulic and coumaric acids (hydroxycinnamic acids), gallic acid, vanillic acid, syringic acid, and hydroxybenzoic acid, among others. Soluble phenolic compounds are well known for their antioxidant properties, which may confer health benefits. These compounds can be isolated via ion exchange chromatography, adsorption methods, ultra- and nano-filtration, and other separation methods known in the art (Kammerer and Carle, “Resin Adsorption and Ion Exchange to Recover and Fractionate Polyphenols”, in Polyphenol in Plants, 2014, Elsevier). Saska and Chou, (“Antioxidants: An Excellent Phytochemical Function Food from Sugarcane”, presented at the 2006 meeting of Sugar Industry Technologists in LaBaule, France) teach a two-step process to purify antioxidant compounds from sugarcane juice. The sugarcane syrup is clarified to remove suspended solids, then fed through an adsorbent column containing a non-ionic polymeric adsorbent such as XAD 1180 (Rohm and Haas) or the Tulsion ADS 600 and 700 resins (Thermax). The eluent is then fed to a strong acid cation exchange resin, such as the Rohm and Haas IR 200 and Purolite C-150. A resulting deashed solution of the anti-oxidant product stream can be concentrated by reverse osmosis or evaporation. The product may contain a lot of color (common for streams containing lignin-derived extractives), but has particularly high antioxidant activity based upon measurements of oxygen radical absorbance capacity.

Kammerer and Carle discuss purification of various phenolic compounds and pigments from plant extracts, including quercetin, caffeic acid, chlorogenic acid, hydroxycinnamates, luteolin, anthocyanins, isoflavones, and catechin. The adsorbent and ion exchange principles proved to be widely transferrable to a variety of compounds of sources of plant extracts, with some variation in resin lifespan and capacity.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. A method of extracting mixed sugars from a lignocellulosic feedstock, comprising:
a. adding water to the feedstock in a cook vessel to produce a feedstock solids and water mixture;
b. heating the mixture in the cook vessel under pressure to an extraction temperature of approximately 130° C. to 180° C. and retaining the extraction temperature and pressure for a retention time of from approximately 30 minutes to approximately 8 hours, wherein, the volume of water combined with the feedstock in the cook vessel is predetermined to keep the water primarily in a liquid state during the retention time, whereby, contents of the vessel comprise a liquid fraction and a solids fraction; and
c. separating the liquid fraction from the solids fraction remaining in the cook vessel to produce a liquid stream and a solids stream,
whereby, the liquid stream comprises a mixture of solubilized fructose, glucose, xylo-oligosaccharides and xylose and the solids stream comprises cellulose and lignin.

2. The method of claim 1, wherein the lignocellulosic feedstock is selected from a group consisting of sugar cane, energy cane, sugar beets, sweet sorghum, fiber sorghum, miscane or mixtures thereof.

3. The method of claim 1, wherein the feedstock comprises sucrose from at least 1 wt % to approximately 30 wt %.

4. The method of claim 1, further wherein the mixture has a pH value of about 3 to about 8 prior to heating.

5. The method of claim 1, wherein the water added to the cook vessel is preheated.

6. The method of claim 1, wherein the cook vessel is pressurized at saturation pressure conditions during the retention time.

7. The method of claim 1, wherein the cook vessel is pressurized at above saturation pressure conditions during the retention time.

8. The method of claim 1, wherein the liquid stream further comprises components selected from group consisting of sucrose, biomass extractives, soluble lignin-derived compounds or bioactive compounds.

9. The method of claim 8, wherein the soluble lignin-derived compounds comprise polyphenols, tannins, flavonoids, anthocyanins, isoflavones, catechins, and phenolic acids.

10. The method of claim 1, wherein the solids stream further comprises residual amounts of components also found in the liquid stream.

11. The method of claim 1, further wherein, after separation, said liquid stream is processed or treated by a method selected from the group consisting of purification, hydrolysis, fermentation and catalysis.

12. A product produced by the method of claim 11.

13. The method of claim 1, further wherein, after separation the resulting solids stream is processed or treated by a method that comprises activation and hydrolysis of cellulose.

14. A product produced by the method of claim 13.

15. A method of extracting mixed sugars from a lignocellulosic feedstock, comprising:
a. physically processing the feedstock to reduce the particle size and increase the surface area of the feedstock;
b. separating the processed feedstock from any liquid that formed during physical processing to produce feedstock solids;
c. combining the processed feedstock with a volume of water in a cook vessel to produce a processed feedstock solids and water mixture;
d. heating the mixture in the cook vessel under pressure to an extraction temperature of approximately 130° C. to 180° C. and retaining the extraction temperature and pressure for a retention time of from approximately 30 minutes to approximately 8 hours, wherein, the volume of water combined with the processed feedstock solids in the cook vessel is predetermined to keep the water primarily in a liquid state during the retention time, whereafter, contents of the vessel comprise a liquid fraction and solids fraction; and
e. separating the liquid fraction from the solids fraction remaining in the vessel to produce a liquid stream and a solids stream,
whereby, the liquid stream comprises a mixture of solubilized fructose, glucose, xylo-oligosaccharides and xylose, and the solids stream comprises cellulose and lignin.

16. The method of claim 15, wherein the lignocellulosic feedstock is selected from a group consisting of sugar cane, energy cane, sugar beets, sweet sorghum, fiber sorghum, miscane and mixtures thereof.

17. The method of claim 15, wherein the feedstock comprises sucrose from at least 1 wt % to approximately 30 wt %.

18. The method of claim 15, further wherein the mixture has a pH value of approximately 3 to approximately 8 prior to heating.

19. The method of claim 15, wherein the water added to the cooking vessel is preheated.

20. The method of claim 15, wherein the cook vessel is pressurized at saturation pressure conditions during the retention time.

21. The method of claim 15, wherein the cook vessel is pressurized at above saturation pressure conditions during the retention time.

22. The method of claim 15, wherein the liquid stream further comprises components selected from group consisting of sucrose, biomass extractives, soluble lignin-derived compounds and bioactive compounds.

23. The method of claim 15, wherein the soluble lignin-derived compounds comprise polyphenols, tannins, flavonoids, anthocyanins, isoflavones, catechins, and phenolic acids.

24. The method of claim 15, wherein the solids stream further comprises residual amounts of components also found in the liquid stream.

25. The method of claim 15, further wherein a volume of water is added to the intact feedstock prior to or during physical processing.

26. The method of claim 15, wherein the physical processing is selected from a group of methods consisting of milling, cutting, pressing, refining and crushing of the feedstock.

27. The method of claim 15, further wherein after separation said liquid stream is processed by a method selected from the group consisting of purification, concentration, hydrolysis, fermentation and catalysis.

28. A product produced by the method of claim 27.

29. The method of claim 15, further wherein, after separation, the solids stream is processed or treated by a method that comprises pretreating and hydrolysis of cellulose.

30. A product produced by the method of claim 29.

31. A component of the liquid stream produced in the method of claim 1.

32. A component of the liquid stream produced in the method of claim 15.

33. A component of the solids stream produced in the method of claim 1.

34. A component of the solids stream produced in the method of claim 15.

35. A method of extracting mixed sugars from a lignocellulosic feedstock, comprising:
a. physically processing the feedstock to reduce the particle size and increase the surface area of the feedstock;
b. adding water to the feedstock in a cook vessel to create a slurry mixture;
c. heating the slurry mixture under pressure to an extraction temperature of approximately 120° C. to 150° C. and retaining the extraction temperature and pressure for approximately 40 minutes, wherein, the volume of water added to the feedstock in the cook vessel is predetermined to keep the water primarily in a liquid state during the retention time, and whereafter, contents of the vessel comprise a liquid portion and solids portion;
d. draining and holding the liquid portion from the cook vessel and retaining the solids portion in the vessel;
e. adding water to the cook vessel;
f. heating and maintaining the temperature and pressure in the cook vessel at approximately 160° C. to 180° for approximately 60 minutes, whereafter, contents of the vessel comprise a second liquid fraction and a solids fraction; and
g. separating the second liquid fraction from the solids fraction remaining in the cook vessel and combining the second liquid fraction with the first liquid portion to produce a liquid stream and a solids stream,
whereby, the liquid stream comprises a mixture of solubilized sucrose, fructose, glucose, xylo-oligosaccharides and xylose.

36. The method of claim 35, wherein the lignocellulosic feedstock is selected from a group consisting of sugar cane, energy cane, sugar beets, sweet sorghum, fiber sorghum, miscane and mixtures thereof.

37. The method of claim 35, wherein the feedstock comprises sucrose from at least 1 wt % to approximately 30 wt %.

38. The method of claim 35, further wherein the mixture has a pH value of approximately 3 to approximately 8 prior to heating.

39. The method of claim 35, wherein the water added to the cook vessel is preheated.

40. The method of claim 35, wherein the cook vessel is pressurized at saturation pressure conditions during the retention time.

41. The method of claim 35, wherein the cook vessel is pressurized at above saturation pressure conditions during the retention time.

42. The method of claim 35, wherein the liquid stream further comprises components selected from group consisting of sucrose, biomass extractives, soluble lignin-derived compounds and bioactive compounds.

43. The method of claim 35, wherein the soluble lignin-derived compounds comprise polyphenols, tannins, flavonoids, anthocyanins, isoflavones, catechins, and phenolic acids.

44. The method of claim 35, wherein the solids stream further comprises residual amounts of components also found in the liquid stream.

45. The method of claim 35, further wherein after separation the resulting liquid stream is processed by a method selected from the group consisting of purification, concentration, hydrolysis, fermentation and catalysis.

46. A product produced by the method of claim 45.

47. The method of claim 35, further wherein after separation the resulting solids stream is processed or treated by a method that comprises pretreating and hydrolysis of cellulose.

48. A product produced by the method of claim 47.

49. A component of the liquid stream produced in the method of claim 42.

50. A component of the solids stream produced in the method of claim 43.
